(19) United States
(12) Patent Application Publication
(10) Pub. No.: US 2018/0282243 A1
(43) Pub. Date: Oct. 4, 2018

(54) SELECTIVE PARTIAL HYDROGENATION OF TERPENES USING A NICKEL-BASED CATALYST

(71) Applicant: TOTAL RAFFINAGE CHIMIE, Courbevoie (FR)

(72) Inventors: Chloé THIEULEUX, Villeurbanne (FR); Emmanuel LACÔTE, Paris (FR); Delphine CROZET, Villeurbanne (FR)

(73) Assignees: CENTRE NATIONAL DE LA RECHERCHE SCIENTIFIQUE (CNRS ), Paris (FR); TOTAL RAFFINAGE CHIMIE, Courbevoie (FR)

(21) Appl. No.: 15/571,372

(22) PCT Filed: May 6, 2016
(86) PCT No.: PCT/EP2016/060154

§ 371 Date: Nov. 2, 2017

(30) Foreign Application Priority Data
May 7, 2015 (EP) 15305704.7

(51) Int. Cl. C07C 5/05; C07C 11/21; B01J 31/16; B01J 31/22; B01J 21/08

(52) U.S. Cl. CPC C07C 5/05; C07C 11/21; B01J 31/1608; B01J 31/2273; B01J 2531/847; C07C 2521/08; C07C 2531/12; C07C 2531/22; B01J 2231/645; B01J 21/08

(57) ABSTRACT

A process for the selective partial hydrogenation of conjugated diene compounds includes at least one, preferably terminal, diene function and at least one additional carbon-carbon double bond, the process including reacting the conjugated diene compounds with hydrogen in the presence of a nickel-NHC based catalyst. The disclosure also relates to a reaction mixture that can be obtained at the end of the process and to a catalyst that can be used in the process. The disclosure also relates to the use of the reaction mixture.

### CROSS-REFERENCE TO RELATED APPLICATIONS

This application is a National Phase Entry of International Patent Application No. PCT/EP2016/060154, filed on May 6, 2016, which claims priority to European Patent Application Serial No. 15305704.7, filed on May 7, 2015, both of which are incorporated by reference herein.

### TECHNICAL FIELD

The invention relates to a process for the selective partial hydrogenation of conjugated diene compounds having at least one conjugated diene function and at least one additional carbon-carbon double bond in order to produce partially hydrogenated compounds. The invention also relates to reaction mixtures that can be obtained at the end of the process of the invention and to a catalyst that can be used in the process of the invention. The invention also relates to the use of the reaction mixtures of the invention.

### BACKGROUND

Olefins can be used as raw materials in different processes. Depending on the processes, different olefins may be used. For example, alpha-olefins can be easily functionalized and used in different industrial processes. Mono-olefins, di-olefins or tri-olefins may be useful as raw materials in different processes, in particular in different kinds of reactions.

As a result of the increasing scarcity of fossil resources and of ever-increasing environmental concerns, the use of molecules derived from biomass is increasingly sought to replace molecules of fossil origin. Up to now, molecules of fossil origin are widely used in the production of fuels or technical fluids, such as lubricants, drilling fluids or solvents. There is now a continuous trend to manufacture fuels and technical fluids thanks to molecules derived from biomass, such as terpenes.

There is a need for renewable olefinic feedstocks that are not derived from fossil fuels. Furthermore, there is a need for alternate olefinic feedstocks, in particular olefinic feedstocks that do not include detectable amounts of sulfur or aromatic compounds. Additionally, there is a need for methylated olefinic feedstocks, in particular olefinic feedstocks in which the methylation position is controlled. In the polymerization technologies, there is also a need to find alternatives, potentially bio-sourced, for replacement of polyisoprenes as well as for the chain transfer agent isoamylene.

Partial hydrogenation of olefinic feedstocks, in particular renewable olefinic feedstocks, allows manufacturing different olefins, such as mono-olefins, di-olefins or tri-olefins, which may subsequently be used as raw materials in different industrial processes. The partial hydrogenation should be selective in order to control the obtained composition and facilitate the separation of the partially hydrogenated compounds that may be made after the partial hydrogenation reaction. There is thus a need for the selective hydrogenation of terpene.

The selective hydrogenation of myrcene has been reported with complexes of ruthenium, chromium, iridium and rhodium. One neutral iridium complex [IrCl(CO)(PPh₃)₂] is described as active for the hydrogenation of myrcene (Journal of Molecular Catalysis A: Chemical 239 (2005) 10-14). Said publication does not disclose nickel catalysts.

Document WO 2012/141783 describes the manufacture of partially hydrogenated molecules from conjugated alkenes. However this document allows obtaining a mixture of mono-, di- or tri-hydrogenated molecules and among them all the isomers for each molecular mass are formed. For example, for the farnesene, the process disclosed in this document leads to a reaction mixture comprising several isomers of molecular mass 206, several isomers of molecular mass 208 and several isomers of molecular mass 210. There is thus a need for a process leading to partially hydrogenated products with an improved selectivity.

Selective hydrogenation of poly-unsaturated alkenes such as farnesene can be highly difficult using either classical heterogeneous or homogeneous catalysts, since the hydrogenation is very active or only allows obtaining a mixture of products with competitive isomerization reaction. Among selective catalyst complexes, most often used complexes are homogenous ones. However, deactivation processes can be reported, such as thermal stability issues for nickel catalysis. One solution is the isolation of transition metal complexes on silica surface.

Document WO 2009/092814 describes organometallic materials that can be used as heterogeneous catalyst. This document does not disclose the selective partial hydrogenation of conjugated diene compounds. Additionally, this document does not disclose a nickel-NHC based catalyst. Organometallics 2012, 31, 806-809 discloses a poorly active homogeneous nickel-NHC catalytic system to hydrogenate cyclopentene with highly hindered NHC ligand.

### SUMMARY

A first object of the present invention is a process for the partial hydrogenation of conjugated diene compounds comprising at least one conjugated diene function and at least one additional carbon-carbon double bond, said process comprising reacting the conjugated diene compounds with hydrogen in the presence of a Nickel-NHC based catalyst, to produce a reaction mixture comprising partially hydrogenated compounds, a portion of said partially hydrogenated compounds resulting from the mono-hydrogenation of one carbon-carbon double bond of the conjugated diene function. According to a preferred embodiment, the at least one conjugated diene function of the conjugated diene compounds is a terminal conjugated diene function. According to an embodiment of the invention, the conjugated diene compounds comprising at least one conjugated diene function and at least one additional carbon-carbon double bond are selected from terpenes, preferably from myrcene and farnesene.

According to an embodiment of the invention, the hydrogenation is performed at a temperature ranging from 10 to 140° C., preferably from 20 to 130° C., more preferably from 40° C. to 120° C., even more preferably from 60 to 110° C. According to an embodiment of the invention, the hydrogenation is performed at a pressure ranging from 2 bars to 35 bars, preferably from 10 bars to 30 bars. According to an embodiment of the invention, the partially hydrogenated compounds comprise at least 50% by weight, preferably at least 60% by weight, more preferably at least 70% by weight of mono-hydrogenated compounds based on the total weight of the partially hydrogenated compounds.

According to an embodiment of the invention, the hydrogenation is performed in the presence of potassium hexamethyldisilazide or of an amine of general formula NQ₃, with Q an alkyl group having from 1 to 12 carbon atoms. Preferably, the partially hydrogenated compounds comprise at least 80% by weight, preferably at least 90% by weight, more preferably at least 95% by weight of mono-hydrogenated compounds based on the total weight of the partially hydrogenated compounds.

According to an embodiment of the invention,
the conjugated diene compounds have the following formula (g):

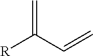

in formula (g), R is a hydrocarbyl radical having 1 to 40 carbon atoms and comprising at least one carbon-carbon double bond, optionally comprising one or more heteroatoms, such as nitrogen, oxygen or sulphur,
and the reaction mixture comprises compounds of formula (g1), compounds of formula (g3), compounds of formula (g4) and compounds of formula (g5), said compounds being obtained from the mono-hydrogenation of the conjugated diene compounds,

wherein:

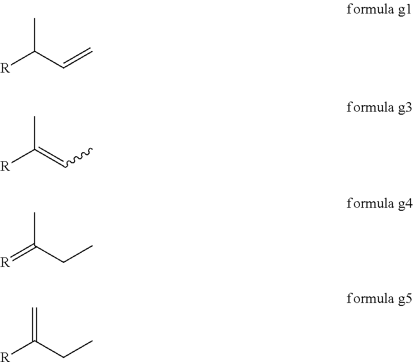

in formulas (g1), (g3) and (g5), R has the same meaning as in formula (g) and in formula (g4), R′ is derived from R with one hydrogen atom in less.

According to an embodiment of the invention, the conjugated diene compounds are farnesenes and the partially hydrogenated compounds comprise mono-hydrogenated compounds in the following amounts:
5 to 15% by weight of compounds of formula (f1),
20 to 40% by weight of compounds of formula (f3),
30 to 60% by weight of compounds of formula (f4),
12 to 20% by weight of compounds of formula (f5),

based on the total weight of the partially hydrogenated compounds,

wherein:

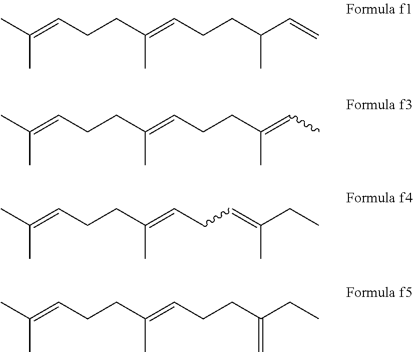

According to an embodiment of the process, the nickel-NHC based catalyst is a homogeneous catalyst. Preferably, the homogeneous catalyst is a complex of formula (I):

[Ni(L1)(L2)NHC(L3)] (I)

wherein

L1, L2 and L3 are independently to each other a ligand, preferably chosen from halogen, NHC, 1,5-cyclooctadiene (COD).

According to an embodiment of the invention, the nickel-NHC based catalyst is a heterogeneous catalyst. Preferably, the heterogeneous catalyst is a Nickel-NHC silica-supported catalyst responding to the following formula (II):

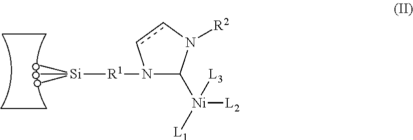

wherein:

L₁, L₂ and L₃ are independently to each other a ligand, preferably selected from halogen, 1,5-cyclo-octadiene (COD), solvent molecule or an interaction with the silica surface,

R¹ is chosen from alkylene or arylene group, optionally substituted,

R² represents an alkyl or an aryl group optionally substituted.

A second object of the present invention is a reaction mixture obtainable by the process of the invention, said reaction mixture comprises partially hydrogenated compounds comprising:
from 5 to 15% by weight of compounds of formula (g1),
from 20 to 40% by weight of compounds of formula (g3),
from 30 to 60% by weight of compounds of formula (g4),
from 12 to 20% by weight of compounds of formula (g5),

based on the total weight of the partially hydrogenated compounds,

wherein:

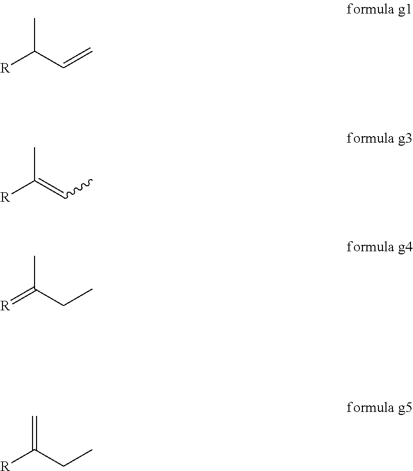

in all the formulas (g1), (g3) and (g5), R is the same group and represents a hydrocarbyl radical having 1 to 40 carbon atoms and comprising at least one carbon-carbon double bond, optionally comprising one or more heteroatoms, such as nitrogen, oxygen or sulphur,
and in formula (g4), R′ is derived from R with one hydrogen atom in less.

According to an embodiment, the reaction mixture of the invention comprises partially hydrogenated compounds comprising:
5 to 15% by weight of compounds of formula (f1),
20 to 40% by weight of compounds of formula (f3),
30 to 60% by weight of compounds of formula (f4),
12 to 20% by weight of compounds of formula (f5),

based on the total weight of the partially hydrogenated compounds,

wherein:

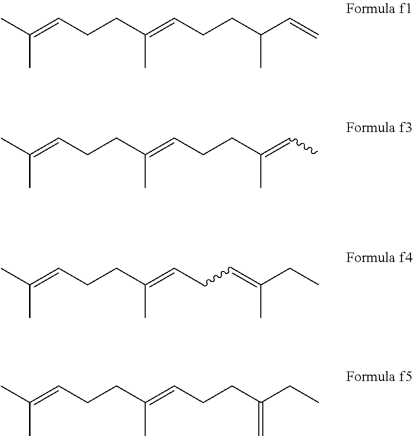

A further object of the invention is the use of the reaction mixture according to the invention or derivatives thereof, in sealants or polymers formulation with silicone, in coating fluids, in metal extraction, in mining, in explosives, in concrete demoulding formulations, in adhesives, in printing inks, in metal working fluids, in resins, in pharmaceutical products, in paint compositions, in polymers used in water treatment, paper manufacturing or printing pastes and cleaning solvents, as cutting fluids, as rolling oils, as EDM (Electronic Discharge Machining) fluids, rust preventive in industrial lubricants, as extender oils, as drilling fluids, as industrial solvents, as viscosity depressants in plasticized polyvinyl chloride formulations, as crop protection fluids.

Another object of the invention is a nickel-NHC silica-supported catalyst that can be used in the process of the invention, said silica-supported catalyst responds to the following formula (II):

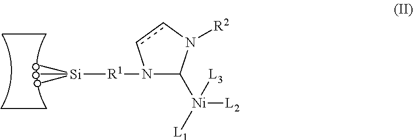

wherein:

the carbon-carbon bond in the NHC cycle can be either a carbon-carbon double bond or a carbon-carbon simple bond, preferably the carbon-carbon bond in the NHC cycle is a carbon-carbon double bond,

L₁, L₂ and L₃ are independently to each other a ligand,

R¹ is chosen from alkylene or arylene group, optionally substituted,

R² represents an alkyl or an aryl group optionally substituted.

Preferably, the catalyst of the invention has the following formula (IIbis):

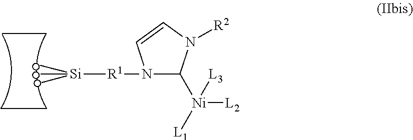

wherein

the carbon-carbon bond in the NHC cycle is a carbon-carbon double bond,

L₁, L₂, L₃, R¹ and R² have the same meaning as in formula (II).

According to an embodiment of the invention, in formulas (II) and/or (IIbis):

L₁, L₂ and L₃ are independently to each other selected from halogen, 1,5-cyclo-octadiene (COD), solvent molecule or represent an interaction with the silica surface,

R¹ is selected from the group consisting of C₁₋₂₀-alkylene, C₅₋₂₀-arylene, which can be optionally substituted with one or more moieties selected from the group consisting of C₁₋₁₀-alkoxy, phosphine, sulfonated phosphine, phosphate, phosphinite, arsine, ether, amine, amide, imine, sulfoxide, carboxyl, nitrosyl, pyridine, substituted pyridine, imidazole, substituted imidazole, pyrazine, substituted pyrazine and thioether,

R² is selected from hydrogen, C₁₋₂₀-alkyl, C₅₋₂₀-aryl, which can be optionally substituted with one or more moieties selected from the group consisting of C₁₋₁₀-alkoxy, phosphine, sulfonated phosphine, phosphate, phosphinite, arsine, ether, amine, amide, imine, sulfoxide, carboxyl, nitrosyl, pyridine, substituted pyridine, imidazole, substituted imidazole, pyrazine, substituted pyrazine and thioether, oxazoline, substituted oxazoline, pyrazoline, substituted pyrazoline.

The process of the invention is simple and allows providing desired products with a high selectivity. Further features and advantages of the invention will appear from the following description of embodiments of the invention, given as non-limiting examples.

### DETAILED DESCRIPTION

### Process for the Partial Hydrogenation

The present invention is directed to a process for the partial hydrogenation of conjugated diene compounds comprising at least one conjugated diene function and at least one additional carbon-carbon double bond, said process comprising reacting the conjugated diene compounds with hydrogen in the presence of a Nickel-NHC based catalyst, to produce a reaction mixture comprising partially hydrogenated compounds, a portion of said partially hydrogenated compounds resulting from the mono-hydrogenation of one carbon-carbon double bond of the conjugated diene function.

### Conjugated Diene Compounds

The conjugated diene compounds that are hydrogenated according to the process of the invention comprise at least one conjugated diene function and at least one additional carbon-carbon double bond. The at least one conjugated diene function of the conjugated diene compound may be either terminal conjugated diene function or not-terminal conjugated diene function.

The conjugated diene compound may be represented by the following formula (g):

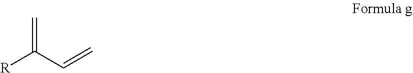

wherein R is a hydrocarbyl radical having 1 to 40 carbon atoms and comprising at least one carbon-carbon double bond, optionally comprising one or more heteroatoms, such as nitrogen, oxygen or sulphur.

Preferably, R is a hydrocarbyl radical having from 5 to 20 carbon atoms and comprising at least one carbon-carbon double bond, optionally comprising one or more heteroatoms, such as nitrogen, oxygen or sulphur. According to a specific embodiment, R consists in carbon and hydrogen atoms.

The conjugated diene compounds may comprise only one kind of conjugated diene compound or a mixture of different conjugated diene compounds. Preferably, the conjugated diene compounds, as starting product of the process, comprise only one kind of conjugated diene compound. The conjugated diene compounds, as starting mixture of the process, generally consist essentially of conjugated diene compounds. Very few impurities may be present in the conjugated diene compounds. Preferably, conjugated diene compounds comprise at least 95% by weight of conjugated diene compounds, more preferably at least 97% by weight, even more preferably at least 99% by weight, based on the total weight of conjugated diene compounds. According to an embodiment, the conjugated diene compounds are chosen from terpenes, preferably from terpenes having from 10 to 40 carbon atoms.

Terpenes are molecules of natural origin, produced by numerous plants, in particular conifers. By definition, terpenes (also known as isoprenoids) are a class of hydrocarbons bearing as the base unit an isoprene moiety (i.e. 2-methyl-buta-1,3-diene). Isoprene [CH₂═C(CH₃)CH═CH₂] is represented below:

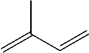

Terpenes may be classified according to the number n (integer) of isoprene units of which it is composed, for example:

n=2: monoterpenes (C₁₀), such as myrcene or pinene (alpha or beta), are the most common;

n=3: sesquiterpenes (C₁₅), such as farnesene;

n=4: diterpenes (C₂₀);

n=5: sesterpenes (C₂₅);

n=6: triterpenes (C₃₀), such as squalene;

n=7: tetraterpenes (C₄₀), such as carotene (C₄₀H₆₄), which is an important pigment of plant photosynthesis.

Many isomers exist in each of the families. The carbon backbone of terpenes may consist of isoprene units arranged end to end to form linear molecules. The arrangement of the isoprene units may be different to form a branched or cyclic backbone.

Preferably, terpenes are chosen from myrcene and farnesenes, preferably from farnesenes, in particular from beta-farnesene. Beta-farnesene refers to a compound having the following formula (f):

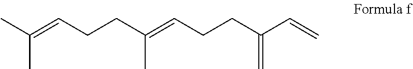

Myrcene refers to a compound having the following formula (m):

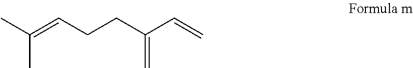

As another example of the conjugated diene compound responding to formula (g), mention may be made of farnesene epoxide:

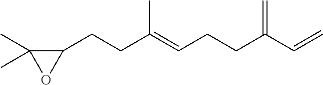 [SMILES: C=CC(=C)CC/C=C(\C)CCC1OC1(C)C]

### Catalyst Used in the Process for the Hydrogenation

The catalyst used in the present invention in order to perform the selective hydrogenation reaction is chosen from Nickel-NHC based catalysts, and some of them are new products per se as explained hereinafter. According to the present invention, NHC refers to a N-heterocyclic carbene and corresponds to a 1,3-di-substituted-imidazol-2-ylidene (R¹R²Im).

In particular, NHC responds to the following formula:

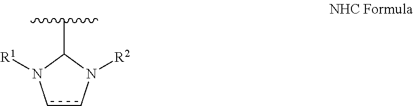

wherein,

the free valence (symbolized by [symbol] ) of the NHC is linked to the metal atom of the catalyst,

the carbon-carbon bond in the NHC cycle can be either a carbon-carbon double bond or a carbon-carbon simple bond, preferably the carbon-carbon bond in the NHC cycle is a carbon-carbon double bond,

R¹ and R² represent independently to each other, an alkyl or an aryl group optionally substituted.

Preferably, R¹ and R² are, independently to each other, selected from the group consisting of C₁₋₂₀-alkyl, C₅₋₂₀-aryl, which can be optionally substituted with one or more moieties selected from the group consisting of C₁₋₁₀-alkoxy, phosphine, sulfonated phosphine, phosphate, phosphinite, arsine, ether, amine, amide, imine, sulfoxide, carboxyl, nitrosyl, pyridine, substituted pyridine, imidazole, substituted imidazole, pyrazine, substituted pyrazine and thioether. R¹ and/or R² may also represent C₅₋₂₀-aryl substituted with one or more moieties selected from oxazoline, substituted oxazoline, pyrazoline or substituted pyrazoline. The nickel metal center of the nickel-NHC catalyst may have an oxidation degree of 0 or +II.

According to the present invention, the catalyst may be supported or not supported. Indeed, the process according to the present invention may be performed by homogeneous catalysis (i.e. the catalyst is soluble in the reaction medium) or heterogeneous catalysis (i.e. the catalyst is not soluble in the reaction medium). When supported, the support is preferably chosen from silica.

According to an embodiment of the invention, the Ni—NHC based catalyst is a complex of general formula (I):

[Ni(L1)(L2)NHC(L3)] (I)

wherein

L1, L2 and L3 are independently to each other a ligand, preferably chosen from halogen, NHC, 1,5-cyclooctadiene (COD).

In particular, COD may be used as ligands when the nickel metal center has an oxidation degree of 0. According to an embodiment, L1, L2 and L3 are independently to each other chosen from halogen, NHC.

Preferably, the Ni—NHC based catalyst is a complex of general formula (Ibis):

[Ni(L1)(L2)(NHC)(NHC)] (Ibis)

wherein

L1 and L2 are independently to each other a ligand, preferably chosen from halogen, 1,5-cyclooctadiene (COD),

both NHC ligands of the above-defined complex may be identical or different, i.e. R¹ and R² as defined above in the NHC definition may be the same or different between both NHC ligands of the complex. Both NHC ligands can be linked together by an alkylene chain, for example by a C₁-C₃ alkylene chain, such as methyl, ethyl or propyl.

According to another embodiment, the Ni—NHC based catalyst is a silica-supported catalyst and responds to the following formula (II):

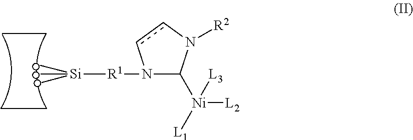

wherein

L₁, L₂ and L₃ are independently to each other a ligand,
R¹ represents a divalent linker, R¹ may be chosen from an alkylene or an arylene group, optionally substituted,

and R² represents an alkyl or an aryl group optionally substituted. Preferably, R¹ is selected from the group consisting of C₁₋₂₀-alkylene, C₅₋₂₀-arylene, which can be optionally substituted with one or more moieties selected from the group consisting of C₁₋₁₀-alkoxy, phosphine, sulfonated phosphine, phosphate, phosphinite, arsine, ether, amine, amide, imine, sulfoxide, carboxyl, nitrosyl, pyridine, substituted pyridine, imidazole, substituted imidazole, pyrazine, substituted pyrazine and thioether, oxazoline, substituted oxazoline, pyrazoline or substituted pyrazoline.

Preferably, R² is selected from the group consisting of C₁₋₂₀-alkyl, C₅₋₂₀-aryl, which can be optionally substituted with one or more moieties selected from the group consisting of C₁₋₁₀-alkoxy, phosphine, sulfonated phosphine, phosphate, phosphinite, arsine, ether, amine, amide, imine, sulfoxide, carboxyl, nitrosyl, pyridine, substituted pyridine, imidazole, substituted imidazole, pyrazine, substituted pyrazine and thioether, oxazoline, substituted oxazoline, pyrazoline or substituted pyrazoline. According to an embodiment, R² may interact with the metal atom, through for example a coordination bond. In particular, if the ligand is weakly coordinated, the ligand may be replaced by the R² radical. As it is well known for the skilled person, the support illustrated in the above formula (II) is a schematic illustration, such that a support comprises one or several metal atoms.

According to an embodiment, L₁, L₂ and L₃ are selected from halogen, 1,5-cyclo-octadiene (COD), solvent molecule or surface interaction. Indeed, the surface of the support (for example the silica) or the solvent may act as a ligand. In particular, the interaction with the surface may be made thanks to the oxygen atoms.

Preferably, in the above-formula (II) of the supported Ni—NHC based catalyst, the carbon-carbon bond in the NHC cycle is a carbon-carbon double bond (which corresponds to the catalyst of formula (IIbis) as defined below). The silica-supported catalyst of formula (II) may be obtained according to a process described hereinafter for the silica-supported catalyst of formula (IIbis).

### Hydrogenation Process

The process of the present invention comprises a step of contacting the conjugated diene compounds with hydrogen in the presence of a specific catalyst, said conjugated diene compounds comprise at least one terminal conjugated diene function and at least one additional carbon-carbon double bond. Preferably, the hydrogenation process is a one-step process, in particular said one-step process consists in the following: mixture of reactants, hydrogenation reaction and recovery of the reaction products. The process of the present invention leads to a reaction mixture comprising mono-hydrogenated compounds, said mono-hydrogenated compounds being compounds wherein one carbon-carbon double bond of the conjugated diene function has been hydrogenated.

By mono-hydrogenated compound, it is to be understood a compound wherein only one carbon-carbon double bond has been hydrogenated.
By di-hydrogenated compound, it is to be understood a compound wherein two carbon-carbon double bonds have been hydrogenated.
By tri-hydrogenated compound, it is to be understood a compound wherein three carbon-carbon double bonds have been hydrogenated.

By “reaction mixture”, it is to be understood the olefinic mixture that is obtained at the end of the hydrogenation process. The reaction mixture may comprise the partially hydrogenated compounds, conjugated diene compounds that have not reacted, fully hydrogenated compounds, by-products (i.e. products obtained by side reactions different from a hydrogenation reaction) and an optional solvent.
By “partially hydrogenated compounds”, it is to be understood unsaturated hydrogenated compounds, i.e. hydrogenated compounds comprising at least one carbon-carbon double bond.

By the expression “the reaction mixture mainly comprises compound(s)”, it is to be understood that said compound(s) represents at least 50% by weight, preferably at least 60% by weight, more preferably at least 70% by weight, even more preferably at least 80% by weight, of the total weight of the reaction mixture.
By “the process is selective”, it is to be understood that the process leads to partially hydrogenated compounds comprising in majority mono-hydrogenated compounds.
By the term ‘in majority”, it is to be understood in a proportion of at least 50% by weight, preferably at least 60% by weight, more preferably at least 70% by weight, even more preferably at least 80% by weight.

According to an embodiment of the invention, the partially hydrogenated compounds comprise at least 50% by weight of mono-hydrogenated compounds, preferably at least 60% by weight, more preferably at least 70% by weight, based on the total weight of the partially hydrogenated compounds. The process of the invention is very selective, in particular, it is possible to mainly obtain mono-hydrogenated compounds, i.e. the reaction mixture comprising partially hydrogenated compounds comprises at least 50% by weight of mono-hydrogenated compounds, based on the total weight of the partially hydrogenated compounds. Indeed, the reaction mixture thus obtained mainly comprises mono-hydrogenated compounds. Very few di-hydrogenated compounds or tri-hydrogenated compounds are obtained with the nickel-NHC based catalyst.

According to an embodiment of the invention, when the conjugated diene compound is a compound of formula (g) as previously detailed, the reaction mixture obtained at the end of the process of the invention, comprises compounds of formula (g1), compounds of formula (g3), compounds of formula (g4) and compounds of formula (g5),

wherein

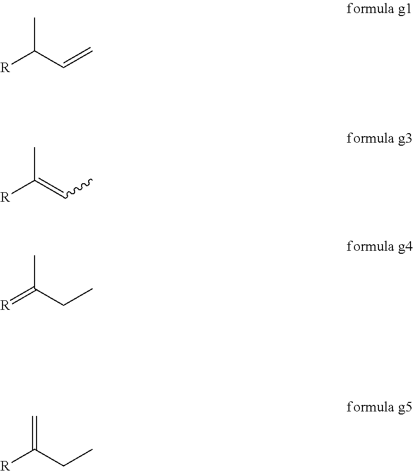

In formulas (g1), (g3) and (g5), R represents the same group as in formula (g). In formula (g4), R′ represents the group R with one hydrogen atom in less (since R′ is linked to the conjugated diene function with a carbon-carbon double bond). Compounds of formula (g1), (g3), (g4) and (g5) are obtained after a mono-hydrogenation of the compounds of formula (g).

Preferably, the partially hydrogenated compounds according to this embodiment comprise:
from 5 to 15% by weight of a compound of formula (g1),
from 20 to 40% by weight of a compound of formula (g3),
from 30 to 60% by weight of a compound of formula (g4),
from 12 to 20% by weight of a compound of formula (g5),

based on the total weight of the partially hydrogenated compounds.

According to an embodiment of the invention, the process is performed at a temperature ranging from 10 to 140° C., preferably from 20 to 130° C., more preferably from 40° C. to 120° C., even more preferably from 60 to 110° C. According to an embodiment of the invention, the process is performed at a hydrogen pressure ranging from 3 bars (3×10⁵ Pa) to 35 bars (35×10⁵ Pa), preferably from 10 bars (10×10⁵ Pa) to 30 bars (30×10⁵ Pa). When the pressure is of 3 bars or less than 3 bars, the hydrogenation process may be performed in a glass reactor. When the pressure is higher than 3 bars, the hydrogenation process is preferably performed in an autoclave.

Hydrogen can be obtained from any source well known by the skilled person. For example, hydrogen can come from reforming of natural gas, gasification of coal and/or biomass, water electrolysis. After production, hydrogen may be purified via a purification step, for example by pressure swing adsorption.

According to an embodiment, the molar ratio between the conjugated diene compounds and the catalyst is from 300 to 10000, preferably from 400 to 8000, more preferably from 500 to 5000. According to an embodiment of the invention, the process is performed in a solvent, such as methanol or toluene, preferably in toluene. Preferably, the amount of solvent is from 10 to 50 mL for an amount of 5 to 40 mmol of conjugated diene compounds. For example, the amount of solvent if about 30 mL for 10 mmol of conjugated diene compounds.

The process according to the invention may be performed in the presence of compound(s) different from the above-detailed compounds, such as potassium hexamethyldisilazide (KHDMS) or an amine. The counter anion of hexamethyldisilazide can be different from potassium, such as lithium or sodium. Other disilazanes may be used, such as H(CH₃)₂Si—N—Si(CH₃)₂H,X (with X=K, Na, Li).

According to an embodiment of the invention, potassium hexamethyldisilazide (KHMDS) or an amine of formula NQ₃, with Q an alkyl group having from 1 to 12 carbon atoms, is added during the reaction of hydrogenation. Preferably, KHMDS or NQ₃ is added in an amount ranging from 0.8 molar equivalents to 5 molar equivalents with respect to NHC ligand. Preferably, KHMDS or NQ₃ is added when the process is performed with a homogeneous catalyst.

KHMDS allows increasing the selectivity of the process towards the mono-hydrogenated compounds. Indeed, according to this embodiment, the reaction mixture may comprise at least 80% by weight of mono-hydrogenated compounds, preferably at least 80% by weight of mono-hydrogenated compounds, more preferably at least 90% by weight of mono-hydrogenated compounds, even more preferably at least 95% by weight of mono-hydrogenated compounds, based on the total weight of the reaction mixture, at the end of the process of the invention. The reaction mixture may then be analyzed according to any methods known by the skilled person, such as by gas chromatography. An analysis by gas chromatography may allow determining the amount of each isomer of the partially hydrogenated compounds present in the reaction mixture.

According to an embodiment of the invention, the conjugated diene compounds are farnesenes. According to this embodiment, the reaction mixture obtained at the end of the process of the invention comprises:
from 5 to 15% by weight of compounds of formula (f1),
from 20 to 40% by weight of compounds of formula (f3),
from 30 to 60% by weight of compounds of formula (f4),
from 12 to 20% by weight of compounds of formula (f5),

based on the total weight of the partially hydrogenated compounds,

wherein:

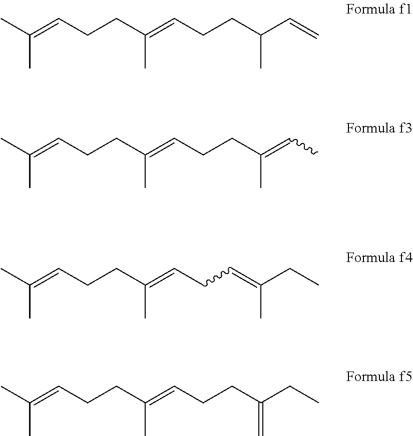

### Reaction Mixture

The present invention also concerns a reaction mixture obtainable by the process of the invention. The reaction mixture of the invention comprises partially hydrogenated compounds comprising:
from 5 to 15% by weight of a compound of formula (g1),
from 20 to 40% by weight of a compound of formula (g3),
from 30 to 60% by weight of a compound of formula (g4),
from 12 to 20% by weight of a compound of formula (g5),

based on the total weight of the partially hydrogenated compounds,

wherein

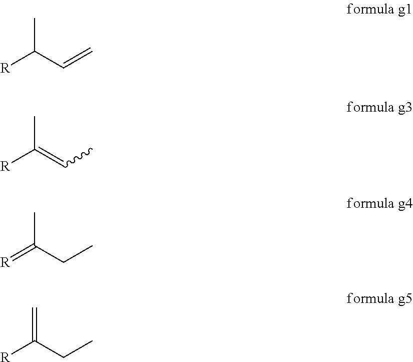

In formulas (g1), (g3) and (g5), R is a hydrocarbyl radical having 1 to 40 carbon atoms and comprising at least one carbon-carbon double bond, optionally comprising one or more heteroatoms, such as nitrogen, oxygen or sulphur. In formula (g4), R′ represents the group R with one hydrogen atom in less (since R′ is linked to the previously conjugated diene function with a carbon-carbon double bond). Preferably, in the above-formulas, R is a hydrocarbyl radical having from 5 to 20 carbon atoms and comprising at least one carbon-carbon double bond, optionally comprising one or more heteroatoms, such as nitrogen, oxygen or sulphur. According to a specific embodiment, R consists in carbon and hydrogen atoms. In one reaction mixture according to the present invention, the R group in each formula (g), (g1), (g3), (g4) and (g5) is identical.

According to an embodiment, the conjugated diene compounds that are hydrogenated according to the process of the invention are farnesenes. According to this embodiment, the reaction mixture according to the invention comprises partially hydrogenated farnesene comprising:
from 5 to 15% by weight of compounds of formula (f1),
from 20 to 40% by weight of compounds of formula (f3),
from 30 to 60% by weight of compounds of formula (f4),
from 12 to 20% by weight of compounds of formula (f5),

based on the total weight of the partially hydrogenated farnesene,

wherein:

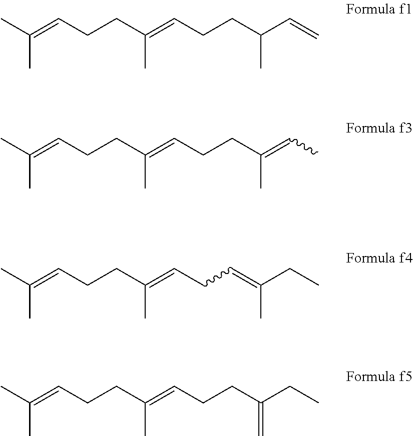

The products contained in the reaction mixture may be further separated and/or purified by any methods known by the one skilled in the art. The reaction mixture of the invention and/or the separated/purified products resulting therefrom, may be used for the preparation of plastics, detergents, lubricants, or oils. In particular, the reaction mixture of the invention may be polymerized, oligomerized, copolymerized or co-oligomerized to make for example an oil, a lubricant or a resin. They may also be functionalized in order to make them suitable for specific applications.

The reaction mixture according to the invention and/or derivatives thereof may be used in sealants or polymers formulation with silicone, in coating fluids, in metal extraction, in mining, in explosives, in concrete demoulding formulations, in adhesives, in printing inks, in metal working fluids, in resins, in pharmaceutical products, in paint compositions, in polymers used in water treatment, paper manufacturing or printing pastes and cleaning solvents, as cutting fluids, as rolling oils, as EDM (Electronic Discharge Machining) fluids, rust preventive in industrial lubricants, as extender oils, as drilling fluids, as industrial solvents, as viscosity depressants in plasticized polyvinyl chloride formulations, as crop protection fluids.

### Catalyst

The present invention also concerns a new catalyst that can be used in the process of the invention. The catalyst of the invention is a silica-supported catalyst and responds to the following formula (II):

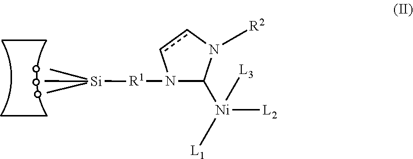

wherein:

the carbon-carbon bond in the NHC cycle can be either a carbon-carbon double bond or a carbon-carbon simple bond, preferably the carbon-carbon bond in the NHC cycle is a carbon-carbon double bond,

L₁, L₂ and L₃ are independently to each other a ligand,

R¹ represents a divalent linker, for example R¹ is chosen from an alkylene or an arylene group optionally substituted,

and R² represents an alkyl or an aryl group optionally substituted. Preferably, R¹ is selected from the group consisting of C₁₋₂₀-alkylene, C₅₋₂₀-arylene, which can be optionally substituted with one or more moieties selected from the group consisting of C₁₋₁₀-alkoxy, phosphine, sulfonated phosphine, phosphate, phosphinite, arsine, ether, amine, amide, imine, sulfoxide, carboxyl, nitrosyl, pyridine, substituted pyridine, imidazole, substituted imidazole, pyrazine, substituted pyrazine and thioether, oxazoline, substituted oxazoline, pyrazoline, substituted pyrazoline.

Preferably, R² is selected from the group consisting of hydrogene, C₁₋₂₀-alkyl, C₅₋₂₀-aryl, which can be optionally substituted with one or more moieties selected from the group consisting of C₁₋₁₀-alkoxy, phosphine, sulfonated phosphine, phosphate, phosphinite, arsine, ether, amine, amide, imine, sulfoxide, carboxyl, nitrosyl, pyridine, substituted pyridine, imidazole, substituted imidazole, pyrazine, substituted pyrazine and thioether, oxazoline, substituted oxazoline, pyrazoline, substituted pyrazoline. According to an embodiment, L₁, L₂ and L₃ are selected from halogen, 1,5-cyclo-octadiene (COD), ethylene glycol dimethylether (DME), solvent molecule or surface interaction. Indeed, the surface of the support (for example the silica) or the solvent may act as a ligand. In particular, the interaction with the surface may be made thanks to the oxygen atoms.

Preferably, the catalyst of the invention has the following formula (IIbis):

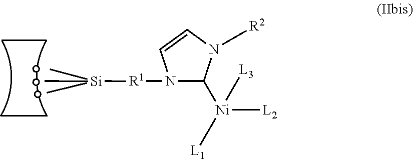

wherein

the carbon-carbon bond in the NHC cycle is a carbon-carbon double bond,

L₁, L₂, L₃, R¹ and R² have the same meaning as in formula (II).

Nickel-based catalysts were found to induce a high selectivity towards mono-hydrogenated products. The catalyst of the invention may be obtained according to the following method. The supported nickel catalyst may be prepared starting from the corresponding imidazolium-functionalized material (M-Im+). The imidazolium-containing material may be synthetized according to methods well known for the skilled person, in particular according to the method described in Tarun K. Maishal et al., Angew. Chem. Int. Ed. 2008, 47, 8654-8656.

In a first step, a triethoxysilane derivative comprising a chlorine atom may react with a sodium iodide in order to form a triethoxysilane derivative comprising an iodine atom:

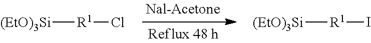

In a second step, there is a step of hydrolysis-polycondensation with tetraethylorthosilicate (TEOS) in the presence for example of a Pluronic® 123 as structure-directing agent:

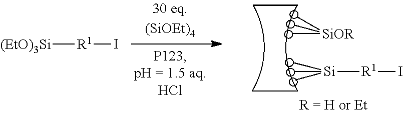

The amount of SiOEt₄ may range from 20 to 200 molar equivalents with respect to (EtO)₃SiR¹I. The above scheme is only illustrative in order to represent a pore of the support. Indeed, another manner of representation may be: IR¹SiO_{1.5}/30SiO₂.

Then, there is a step of functionalization with a derived of imidazole to generate the imidazolium-functionalities:

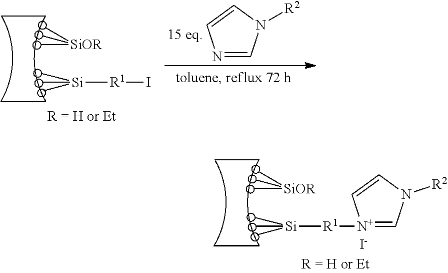

Optionally, if the R group is not a hydrogen group, there may be a step of hydrolysis:

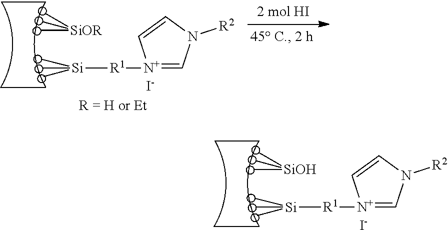

Then, a passivation step may be performed to transform the surface silanols groups into trimethoxysiloxane groups. This step is optional:

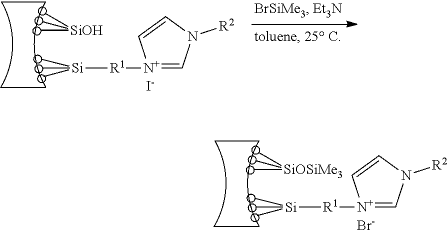

Then, the silica-supported nickel catalyst of the invention may be obtained according to the following procedure. Two synthetic pathways are possible from the imidazolium-functionalized material (M-Im⁺):

(i) preparation of the corresponding material with a supported silver-NHC complex (M-Ag), and then transmetallation reaction to obtain the supported nickel-NHC complex (M-Ni),

(ii) formation of the NHC species by deprotonation of the imidazolium moiety either directly by ligands coordinated to the nickel metal center, or in the presence of an external base.

### Synthetic Pathway (i):

In a further step, the imidazolium-containing material is treated with AgOC(CF₃)₃ to give a silver-NHC supported complex:

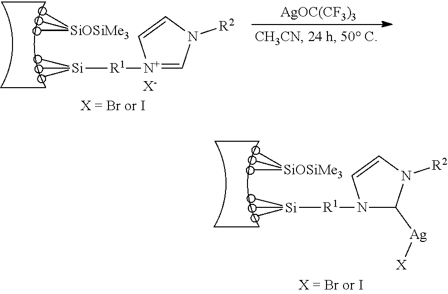

X⁻, depending on the presence or not of the optional passivation step detailed above, may represent either Br⁻ or I⁻.

Finally, the supported nickel-NHC complex according to the invention may then be obtained from the silver-NHC complex by the following reaction conditions:

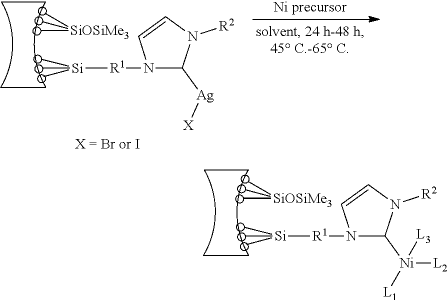

Among nickel precursors, mention may be made of: [Ni(L1)(L2)(L3)], where L₁, L₂, L₃ represent ligands, such as halogen, 1,5-cyclo-octadiene (COD), ethylene glycol dimethylether (DME), solvent molecule or surface interaction. According to an embodiment, the nickel precursors or nickel complexes are selected from [Ni(OAc)₂], [Ni(Br₂)DME], [Ni(Cl₂)DME], [Ni(COD)₂]. One or more nickel precursors may be used.

### Synthetic Pathway (ii):

The imidazolium-containing material may react with a nickel complex according to the following pathway, if the deprotonation occurs directly by ligands coordinated to the nickel center:

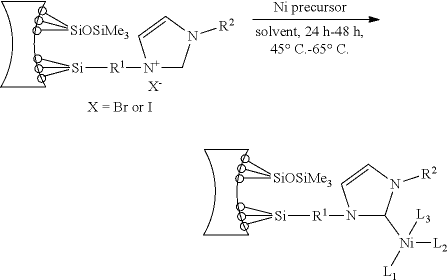

Alternatively, in the synthetic pathway (ii), the deprotonation may occur in the presence of an external base, such as KHMDS in toluene. Among nickel precursors, mention may be made of: [Ni(L1)(L2)(L3)], where L₁, L₂, L₃ represent ligands, such as halogen, 1,5-cyclo-octadiene (COD), ethylene glycol dimethylether (DME), solvent molecule or surface interaction. According to an embodiment, the nickel precursors are selected from [Ni(OAc)₂], [Ni(Br₂)DME], [Ni(Cl₂)DME], [Ni(COD)₂]. One or more nickel precursors may be used. A specific example of manufacture of a catalyst according to the invention is described in the experimental part of the application.

According to an embodiment, the supported catalyst according to the invention has the following characteristics:
The material may exhibit an N₂ adsorption-desorption isotherm at 77 K of type IV, from 300 to 1200 m²/g, for example of 1146 m²/g, which is characteristic of mesoporous materials, with a large BET specific surface area.
The material may have a pore volume (Vp) ranging from 0.5 to 1.5 cm3/g, for example of around 1.4 cm³/g.
The material may also exhibit a mean pore diameter (D_{pBJH}) ranging from 3 to 25 nm, for example of 5.7 nm.
The TEM and powder XRD measurements are consistent with a material having a long-range structuration of the pore network with a 2D hexagonal array. ¹³C solid state NMR spectroscopy confirms the presence of the functional groups. The ²⁹Si NMR spectrums show the characteristic signals corresponding to the organic units bounded to the matrix via three Si—O bonds and to the degree of condensation of the material.
The Nickel-NHC containing materials are classically described by X-ray diffraction, elemental analysis, N₂ adsorption/desorption, TEM and ¹H, ¹³C, and ²⁹Si solid-state NMR spectroscopy.

### EXAMPLES

### Example 1: Preparation of the Supported Catalyst According to the Invention

Step 1: A protected imidazolium-containing material (M-Im⁺) is provided. It may for example be obtained by cocondensation of tetraethylorthosilicate (TEOS) and iodopropyltriethoxysilane (IC₃H₆Si(OEt)₃) in a hydrolytic sol-gel process in the presence of Pluronic 123 as structure-directing agent. This material is then treated with mesitylimidazole to generate the corresponding imidazolium functionalities and then potentially also with Me₃SiBr/NEt₃ to transform the surface silanol groups into trimethylsiloxane moieties. The following component is obtained:

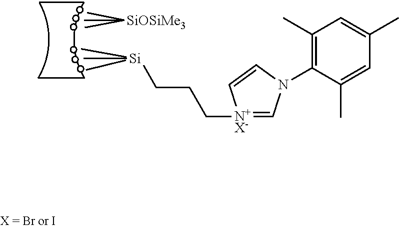

### Preparation According to Synthetic Pathway (i):

Step 2(i): 2.6 g of protected imidazolium containing material M-Im⁺ prepared as previously described (step 1), was mixed with 0.782 g of AgOC(CF₃)₃ under inert atmosphere in the absence of light and all was dissolved in 45 mL of dried and degassed CH₃CN. The mixture was stirred overnight at 25° C. The solid was filtered under argon and washed 3 times by degassed and dried CH₃CN (20 mL) and CH₂Cl₂ (20 mL). The material was dried at 25° C. under vacuum overnight. A brownish powder (2.5 g), of M-AgX (X=Br, I), was obtained. If there is no step of passivation, X=I and if there is a step of passivation, X=Br. The following component is thus obtained:

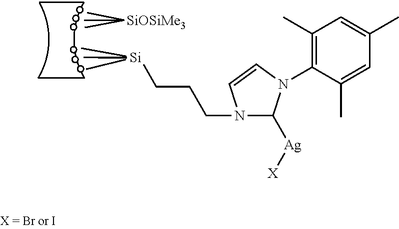

Step 3(i):

400 mg (0.17 mmol) of dried silver material, M-Ag, was mixed with 54.4 mg (0.17 mmol) of [Ni(DME)Br₂] under argon atmosphere, and then dissolved in 20 mL of dried and degassed CH₃CN and left for stirring for 72 h at 65° C. The resulting powder was filtered under argon atmosphere and washed three times with dried and degassed CH₃CN (20 mL). The material was dried at 25° C. under vacuum overnight.

The powder was characterized by TEM, X-ray diffraction and ¹H, ¹³C and ²⁹Si NMR spectroscopy and elemental analysis. The following component is obtained:

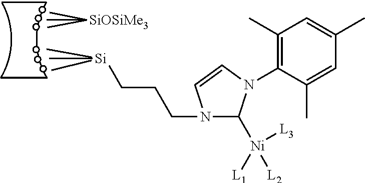

with L₁=halogen such as bromide,

L₂=halogen, such as bromide, or DME or solvent molecule or surface interaction,

L₃=DME or solvent molecule or surface interaction.

DME is a bidentate ligand that can coordinate to the metal center by its two oxygen atoms (in this case, L₂ and L₃ correspond to the DME). Optionally, DME can then either be kept coordinated to the metal center by one oxygen atom or be completely removed and L₃ may be a solvent molecule or a surface interaction.

### Preparation According to Synthetic Pathway (ii):

Step 2(ii): 500 mg (0.23 mmol) of imidazolium material, M-Im⁺, was mixed with 41 mg (0.23 mmol) of [Ni(OAc)₂] under argon atmosphere, and then dissolved in 20 mL of dried and degassed CH3CN and left for stirring for 72 h at 65° C. The resulting powder was filtered under argon atmosphere and washed three times with dried and degassed CH₃CN (20 mL). The resulting powder was dried under vacuum overnight.

The powder was characterized by TEM, X-ray diffraction and ¹H, ¹³C and ²⁹Si NMR spectroscopy and elemental analysis. The following component is obtained:

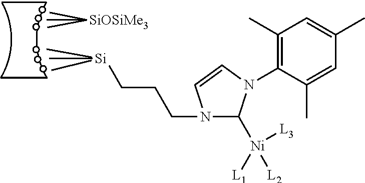

with L₁=halogen such as bromide,

L₂=halogen, such as bromide, or solvent molecule or surface interaction,

L₃=DME or solvent molecule or surface interaction.

DME is a bidentate ligand that can coordinate to the metal center by its two oxygen atoms (in this case, L₂ and L₃ correspond to the DME). Optionally, DME can then either be kept coordinated to the metal center by one oxygen atom or be completely removed and L₃ may be a solvent molecule or a surface interaction.

### Example 2: Hydrogenation Process

The process according to the present invention has been performed using beta-farnesene as conjugated diene compounds.

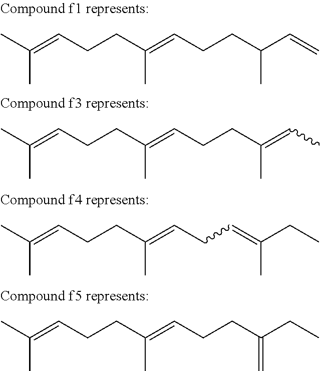

The farnesene conversion refers to the amount in percentage by weight of farnesene that have reacted.

Two different nickel-NHC based catalysts have been tested:
Ex. 2a: a not supported (homogeneous) nickel-bis-NHC catalyst and
Ex. 2b: a supported (heterogeneous) nickel-mono-NHC catalyst.
   All hydrogenation process experiments were performed following the same procedure in a 90 mL stainless-steel autoclave. The catalyst (10 mg) was suspended in β-Farnesene (10 mmol, 2.04 g) and dodecane (5 mmol, 0.85 g) used as internal standard. The mixture was introduced in the reactor, followed by the solvent (Toluene, 30 mL). The closed reactor was then purged three times with the H₂ gas mixture. The reaction mixture was placed under 10 bars H₂ and heated until the desired temperature with a 800 rpm stirring rate. The pressure was then completed until 30 bar H₂. The experiment was running under a continuous feed of gas mixture. Samples were taken during the experiment in order to follow the reaction course by gas chromatography. At the end of the reaction, the autoclave was cooled to room temperature and then slowly depressurized. The crude mixture was analyzed by gas chromatography.

### Ex. 2a: Homogeneous Nickel-Bis-NHC Catalysts

Reaction Conditions/Homogeneous Catalysis:
molar ratio farnesene/nickel=500,
Methanol (MeOH) 30 mL,
temperature range (70° C.-110° C.),
30 bar H₂ pressure in autoclave,
[Ni(OAc)₂], bis-imidazolium salt, with or without KHMDS: Typical example of bis-imidazolium salt: 1,1′-Dimesityl-3,3′-ethylenediimidazolium ditosylate (2-Mes-Mes), and its corresponding complex Ni(2-mes-Mes), or 1,1′-Diethyl-3,3′-ethylenediimidazolium ditosylate (2-Et-Et), and its corresponding complex Ni(2-Et-Et). The in situ generated homogeneous catalysts tested have the following formula:

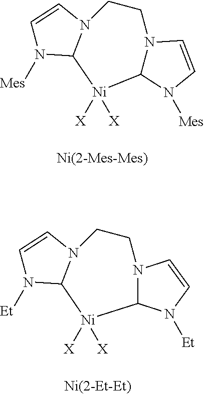

A description of the synthesis of the above homogeneous catalysts is made for example in W. A. Herrmann, J. Schwarz, M. G. Gardiner, M. Spiegler, Journal of Organometallic Chemistry, 1999, 575, 80-86. Selectivities with homogeneous catalysis are indicated in the table 1. The column “isomers 206” refers to the amount of mono-hydrogenated compounds in the reaction mixture. The column “isomers 208” refers to the amount of di-hydrogenated compounds in the reaction mixture. Said amounts are expressed in percentage by weight based on the total weight of the reaction mixture.

The “selectivity/206” column refers to the weight percentage of each mono-hydrogenated compound with respect to the total weight of the partially hydrogenated compounds. The farnesene conversion refers to the amount in percentage by weight of farnesene that have reacted.

TABLE 1
selectivities obtained with homogeneous catalysis

| | Temperature (° C.), | | | | | | |
|---|---|---|---|---|---|---|---|
| | time (hour) | | | | | | |
| | (Farnesene | Isomers | Isomer | Selectivity/206 | | | |
| Conditions | conversion %) | 206 (%) | 208 (%) | f1 | f3 | f4 | f5 |
| Ni(2-Mes-Mes) | 70° C. - 22 h | 77 | 22.9 | traces | 22.9 | 38.1 | 16 |
| | (100%) | | | | | | |
| Ni(2-Mes-Mes)/ | 70° C. - 22 h | >99.9 | <0.1 | 10.8 | 39.7 | 34.3 | 15.2 |
| with KHMDS | (100%) | | | | | | |
| Ni(2-Et-Et) | 70° C. - 22 h | 86.3 | 13.2 | 5.1 | 29.8 | 36.3 | 15.1 |
| | (100%) | | | | | | |
| Ni(2-Et-Et)/ | 70° C. - 22 h | >99.9 | <0.1 | 13.7 | 36.9 | 34.8 | 14.6 |
| with KHMDS | (100%) | | | | | | |

100% of the farnesene have been hydrogenated according to the process of the invention with the homogeneous nickel-NHC based catalyst. Similar results are obtained when the reaction is performed at 110° C. The process of the invention is very selective since it allows obtaining at least 77% by weight of mono-hydrogenated compounds at the end of the process. The amount of mono-hydrogenated compounds may reach almost 100% by weight.

When the conversion of farnesene is 100% and when the partially hydrogenated compounds comprise at least 90% by weight of mono-hydrogenated compounds, then the distribution of the mono-hydrogenated compounds is the following:
10-15% by weight of compounds of formula (f1),
20-40% by weight of compounds of formula (f3),
30-40% by weight of compounds of formula (f4), and
13-16% by weight of compounds of formula (f5),

based on the total weight of the partially hydrogenated compounds.

### Ex. 2b: Supported Nickel-monoNHC Catalysts

Reactions Conditions/Supported Catalyst:
Pressure H₂: 30 bar—autoclave
Temperature 90° C.-110° C.,
Toluene containing traces of MeOH used as solvent,
Molar Ratio farnesene/nickel: minimum 500,
without KHMDS,
the catalyst used is the one obtained in example 1.

Selectivities are indicated in the table 2 below. The column “isomers 206” refers to the amount of mono-hydrogenated compounds in the reaction mixture. The column “isomers 208” refers to the amount of di-hydrogenated compounds in the reaction mixture. Said amounts are expressed in percentage by weight based on the total weight of the reaction mixture.

The “selectivity/206” column refers to the weight percentage of each mono-hydrogenated compound with respect to the weight of the partially hydrogenated compounds. In table 2, experiments A and B illustrate two different temperatures: 90° C. and 110° C. and experiments B and C illustrate the performances of two catalysts of same chemical nature obtained respectively by pathway (i) and pathway (ii).

TABLE 2
selectivities obtained with heterogeneous catalysis

| | | Temperature | Iso- | Iso- | | | | |
|---|---|---|---|---|---|---|---|---|
| | | (° C.), time (h) | mers | mers | | | | |
| | | (Conversion | 206 | 208 | Selectivity/206 | | | |
| | Conditions | Farnesene %) | (%) | (%) | f1 | f3 | f4 | f5 |
| A | Ni- | 90° C. - 25 h - | 98 | 2 | 6 | 23 | 50 | 19 |
| | monoNHC | 68% | | | | | | |
| | supported | | | | | | | |
| | catalyst | | | | | | | |
| | prepared via | | | | | | | |
| | pathway (i) | | | | | | | |
| B | Ni- | 110° C. - 25 h - | 99 | 1 | 5 | 21 | 52 | 21 |
| | monoNHC | 100% | | | | | | |
| | supported | | | | | | | |
| | catalyst | | | | | | | |
| | prepared via | | | | | | | |
| | pathway (i) | | | | | | | |
| C | Ni- | 110° C. - 25 h - | 99 | 1 | 5 | 21 | 52 | 21 |
| | monoNHC | 100% | | | | | | |
| | supported | | | | | | | |
| | catalyst | | | | | | | |
| | prepared via | | | | | | | |
| | pathway (ii) | | | | | | | |

As illustrated in table 2, the process of the invention is also very selective with a heterogeneous catalysis since it allows obtaining a reaction mixture comprising 98% by weight of mono-hydrogenated compounds at the end of the process, based on the total weight of the partially hydrogenated compounds. At the end of the process with the supported Nickel-monoNHC catalyst, the reaction mixture comprises:
5-6% by weight of compounds of formula (f1),
21-23% by weight of compounds of formula (f3),
50-52% by weight of compounds of formula (f4),
19-21% by weight of compounds of formula (f5),

based on the total weight of the partially hydrogenated compounds.

### Ex. 3: Effect of the Molar Ratio Farnesene/Nickel

Reactions Conditions/Supported Catalyst:
Pressure H₂: 30 bar—autoclave
30 mL of toluene and 0.05 mL of MeOH,
without KHMDS,
the catalyst used is the one obtained in example 1 via pathway (ii). Selectivities are indicated in the table 3 below, with the same meaning as in table 2.

TABLE 3
effect of the molar ratio farnesene/nickel

| | | Temperature | | | | | | |
|---|---|---|---|---|---|---|---|---|
| | | (° C.), time (h) | | | | | | |
| | molar ratio | (Conversion | Isomers | Isomers | Selectivity/206 | | | |
| | farnesene/Ni | Farnesene %) | 206 (%) | 208 (%) | f1 | f3 | f4 | f5 |
| D | 1000 | 110° C. - 22 h | 99 | 1 | 5 | 21 | 52 | 21 |
| | | (100%) | | | | | | |
| E | 2000 | 90° C. - 21 h | 98 | 2 | 6 | 23 | 50 | 19 |
| | | (100%) | | | | | | |
| F | 4000 | 110° C. - 21 h | 99 | 1 | 5 | 21 | 52 | 21 |
| | | (100%) | | | | | | |

Table 3 shows that the process provides satisfying selectivities and satisfying conversion for different values of the molar ratio conjugated diene compounds/nickel.

### Ex. 4: Effect of the Solvent

Reactions Conditions/Supported Catalyst:
Pressure H₂: 30 bar—autoclave,
temperature: 90° C.,
without KHMDS.

Experiment H was performed with the catalyst prepared according to example 1 via pathway (i) with 30 mL of toluene and 0.05 mL of MeOH and with a molar ratio farnesene/nickel of 1000. Experiment I was performed with the catalyst prepared according to example 1 via pathway (ii) with 30 mL of toluene and 0.2 mL of MeOH and with a molar ratio farnesene/nickel of 2000. Experiment J was performed with the catalyst prepared according to example 1 via pathway (ii) with 30 mL of toluene and 0.05 mL of MeOH and with a molar ratio farnesene/nickel of 2000. Selectivities obtained for experiments H, I and J are indicated in the table 4 below, with the same meaning as in table 2.

TABLE 4
effect of the solvent

| | time (h) | | | | | | |
|---|---|---|---|---|---|---|---|
| | (Conversion | Isomers | Isomers | Selectivity/206 | | | |
| | Farnesene %) | 206 (%) | 208 (%) | f1 | f3 | f4 | f5 |
| H | 25 h - 68% | 98 | 2 | 6 | 23 | 50 | 19 |
| I | 22 h - 100% | 98 | 2 | 7 | 30 | 41 | 20 |
| J | 21 h - 62% | 96 | 4 | 5 | 20 | 51 | 20 |

Table 3 shows that the process provides satisfying selectivities for different solvents. The conversion is improved when the amount of methanol in the toluene solvent is increased.

1. A process for partial hydrogenation of conjugated diene compounds comprising at least one conjugated diene function and at least one additional carbon-carbon double bond, the process comprising reacting the conjugated diene compounds with hydrogen in the presence of a Nickel-NHC based catalyst, to produce a reaction mixture comprising partially hydrogenated compounds, a portion of the partially hydrogenated compounds resulting from the mono-hydrogenation of one carbon-carbon double bond of the conjugated diene function.

2. The process according to claim 1, wherein the at least one conjugated diene function is a terminal conjugated diene function.

3. The process according to claim 1, wherein the conjugated diene compounds comprising at least one conjugated diene function and at least one additional carbon-carbon double bond are selected from terpenes.

4. The process according to claim 1, wherein the hydrogenation is performed at a temperature ranging from 10 to 140° C.

5. The process according to claim 1, wherein the hydrogenation is performed at a pressure ranging from 2 bars to 35 bars.

6. The process according to claim 1, wherein the partially hydrogenated compounds comprise at least 50% by weight of mono-hydrogenated compounds based on the total weight of the partially hydrogenated compounds.

7. The process according to claim 1, wherein the hydrogenation is performed in the presence of potassium hexamethyldisilazide or of an amine of general formula NQ₃, with Q an alkyl group having from 1 to 12 carbon atoms.

8. The process according to claim 7, wherein the partially hydrogenated compounds comprise at least 80% by weight of mono-hydrogenated compounds based on the total weight of the partially hydrogenated compounds.

9. The process according to claim 1, wherein
the conjugated diene compounds have the following formula (g): 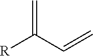
in formula (g), R is a hydrocarbyl radical having 1 to 40 carbon atoms and comprising at least one carbon-carbon double bond,
and the reaction mixture comprises compounds of formula (g1), compounds of formula (g3), compounds of formula (g4) and compounds of formula (g5), the compounds being obtained from the mono-hydrogenation of the conjugated diene compounds,
wherein: 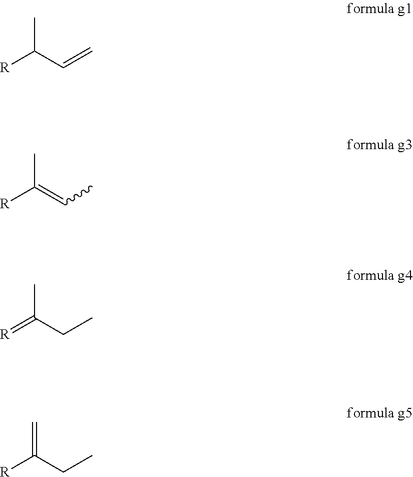
in formulas (g1), (g3) and (g5), R has the same meaning as in formula (g) and in formula (g4), R′ is derived from R with one hydrogen atom in less.

10. The process according to claim 1, wherein the conjugated diene compounds are farnesenes and the partially hydrogenated compounds comprise mono-hydrogenated compounds in the following amounts:
5 to 15% by weight of compounds of formula (f1),
20 to 40% by weight of compounds of formula (f3),
30 to 60% by weight of compounds of formula (f4),
12 to 20% by weight of compounds of formula (f5),
based on the total weight of the partially hydrogenated compounds,
wherein:

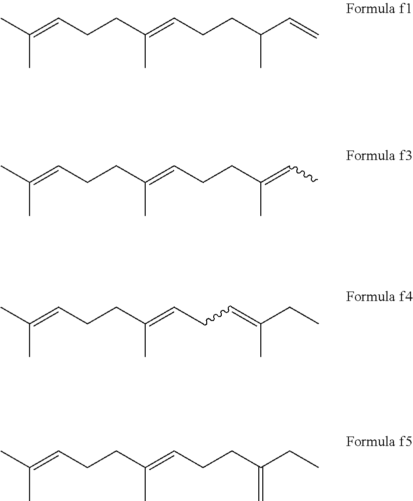

11. The process according to claim 1, wherein the Nickel-NHC catalyst is a homogeneous catalyst.

12. The process according to claim 11, wherein the homogeneous catalyst is a complex of formula (I):
[Ni(L1)(L2)NHC(L3)] (I)
wherein
L1, L2 and L3 are independently to each other a ligand.

13. The process according to claim 1, wherein the Nickel-NHC catalyst is a heterogeneous catalyst.

14. The process according to claim 13, wherein the heterogeneous catalyst is a Nickel-NHC silica-supported catalyst responding to the following formula (II): 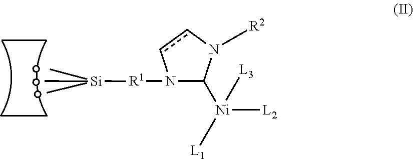
wherein:
L₁, L₂ and L₃ are independently to each other a ligand,
R¹ is chosen from alkylene or arylene group, optionally substituted, and
R² represents an alkyl or an aryl group optionally substituted.

15. A reaction mixture comprising partially hydrogenated compounds comprising:
from 5 to 15% by weight of compounds of formula (g1),
from 20 to 40% by weight of compounds of formula (g3),
from 30 to 60% by weight of compounds of formula (g4),
from 12 to 20% by weight of compounds of formula (g5),
based on the total weight of the partially hydrogenated compounds,
wherein: 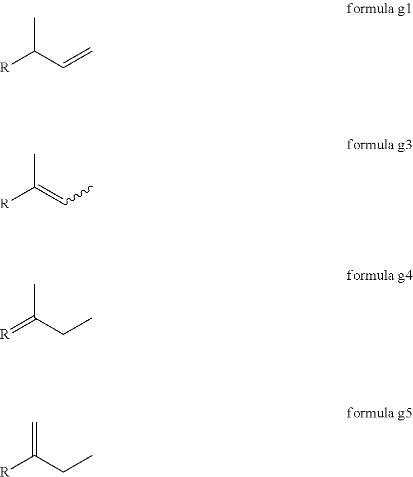
in all the formulas (g1), (g3) and (g5), R is the same group and represents a hydrocarbyl radical having 1 to 40 carbon atoms and comprising at least one carbon-carbon double bond, and
in formula (g4), R′ is derived from R with one hydrogen atom in less.

16. The reaction mixture according to claim 15, comprising partially hydrogenated compounds comprising:
5 to 15% by weight of compounds of formula f1
20 to 40% by weight of compounds of formula f3
30 to 60% by weight of compounds of formula f4
12 to 20% by weight of compounds of formula f5,
based on the total weight of the partially hydrogenated compounds,
wherein:

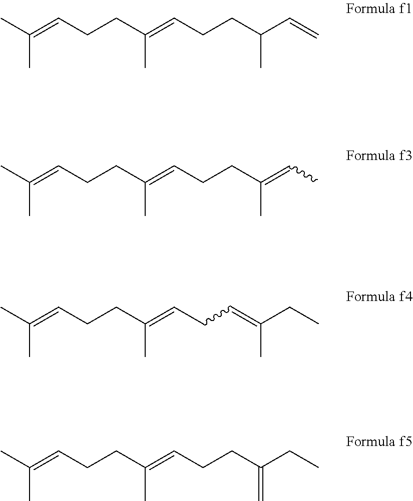

17. The reaction mixture according to any claim 15 or derivatives thereof, in sealants or polymers formulation with silicone, in coating fluids, in metal extraction, in mining, in explosives, in concrete demoulding formulations, in adhesives, in printing inks, in metal working fluids, in resins, in pharmaceutical products, in paint compositions, in polymers used in water treatment, paper manufacturing or printing pastes and cleaning solvents, as cutting fluids, as rolling oils, as Electronic Discharge Machining (EDM) fluids, rust preventive in industrial lubricants, as extender oils, as drilling fluids, as industrial solvents, as viscosity depressants in plasticized polyvinyl chloride formulations, as crop protection fluids.

18. A nickel-NHC silica-supported catalyst responding to the following formula (II): 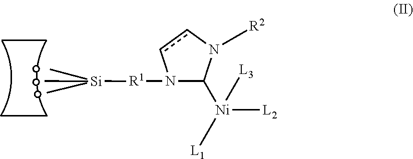
wherein:
L₁, L₂ and L₃ are independently to each other a ligand,
R¹ is chosen from alkylene or arylene group, optionally substituted, and
R² represents an alkyl or an aryl group optionally substituted.

19. The nickel-NHC silica-supported catalyst according to claim 18, having the following formula (IIbis): 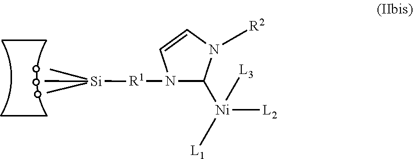
wherein:
L₁, L₂ and L₃ are independently to each other a ligand,
R¹ is chosen from alkylene or arylene group, optionally substituted, and
R² represents an alkyl or an aryl group optionally substituted.

20. The nickel-NHC silica-supported catalyst according to claim 18, wherein:
L₁, L₂ and L₃ are independently to each other selected from halogen, 1,5-cyclo-octadiene (COD), solvent molecule or represent an interaction with the silica surface;
R¹ is selected from the group consisting of C₁₋₂₀-alkylene, C₅₋₂₀-arylene, which can be optionally substituted with one or more moieties selected from the group consisting of C₁₋₁₀-alkoxy, phosphine, sulfonated phosphine, phosphate, phosphinite, arsine, ether, amine, amide, imine, sulfoxide, carboxyl, nitrosyl, pyridine, substituted pyridine, imidazole, substituted imidazole, pyrazine, substituted pyrazine and thioether;
R² is selected from hydrogen, C₁₋₂₀-alkyl, C₅₋₂₀-aryl, which can be optionally substituted with one or more moieties selected from the group consisting of C₁₋₁₀-alkoxy, phosphine, sulfonated phosphine, phosphate, phosphinite, arsine, ether, amine, amide, imine, sulfoxide, carboxyl, nitrosyl, pyridine, substituted pyridine, imidazole, substituted imidazole, pyrazine, substituted pyrazine and thioether, oxazoline, substituted oxazoline, pyrazoline, substituted pyrazoline.
